(19) United States
(12) Patent Application Publication
(10) Pub. No.: US 2023/0200984 A1
(43) Pub. Date: Jun. 29, 2023

(54) Aortic Root Replacement Device

(71) Applicant: Fondation Hopital Saint Joseph, Paris (FR)

(72) Inventors: Stephan Haulon, Paris (FR); Dominique Fabre, Garches (FR); Aurelien Vallee, Paris (FR)

(73) Assignee: Fondation Hopital Saint Joseph, Paris (FR)

(21) Appl. No.: 17/926,820

(22) PCT Filed: May 20, 2021
(86) PCT No.: PCT/FR2021/050916

§ 371 Date: Nov. 21, 2022

(30) Foreign Application Priority Data
May 20, 2020 (FR) FR2005214

(51) Int. Cl. A61F 2/24

(52) U.S. Cl. CPC A61F 2/2418; A61F 2002/828

(57) ABSTRACT

An aortic root replacement device is movable between a deployed configuration and a retracted configuration. The device includes an intraventricular stent having a proximal portion and a distal portion, the intraventricular stent is flared from the proximal portion towards the distal portion such that when the device is deployed in the ventricle below the aortic valve it is held in position in the ventricle. The device also includes—an aortic valve unit and an aortic stent extending from the intraventricular stent. The intraventricular stent, the aortic valve unit and the aortic stent form a unitary assembly in retracted configuration and in deployed configuration, the aortic stent includes at least three means for connecting the device to the coronary arteries.

### GENERAL TECHNICAL FIELD

The invention relates to implantable medical devices and more particularly relates to medical stents used in cardiology or vascular surgery. And the invention relates more specifically to an endoprosthesis for the replacement of the aortic root by the endovascular route with proximal sub-aortic sealing.

### PRIOR ART

An organism (human or animal) with a heart and an aortic root can be affected by several pathologies such as type A aortic dissection, aneurysmal disease, pseudoaneurysms, infectious pathologies.

These infections may require repair or replacement of the aortic root, which is usually performed by open-heart surgery with the need for cardiopulmonary bypass.

The aortic root is a portion of the aorta and an aortic valve located at the heart. From the aortic valve emerges the aorta, hence the name of this area by aortic root. In this area, located at the heart emerge, the right and left coronaries and the ascending aorta which extends along the aortic arch.

Surgery aiming at replacing or repairing the aortic root is often an open surgery which, however, has significant complication and mortality rates. Currently, 20% of patients with acute type A dissection are declared ineligible for surgery, especially elderly patients.

These patients may, however, be eligible for aortic root replacement using endovascular surgery.

### PRESENTATION OF THE INVENTION

A purpose of the invention is to have a vascular endoprosthesis allowing endovascular replacement of the aortic root without recourse to open-heart surgery.

To this end, the invention proposes, according to a first aspect, an aortic root replacement device that is movable between a deployed configuration and a retracted configuration, said device comprising:
an intraventricular stent comprising a proximal portion and a distal portion, said intraventricular stent being flared from the proximal portion towards the distal portion such that when the device is deployed in the ventricle it is held in position in the ventricle;
an aortic valve unit;
an aortic stent extending from the intraventricular stent;

the intraventricular stent, the aortic valve unit and the aortic stent forming a unitary assembly in retracted configuration and in deployed configuration.

The invention, according to the first aspect, is advantageously completed by the following features, taken alone or in any of their technically possible combination:
the aortic stent extends from the intraventricular stent and comprises a tubular portion;
the aortic valve unit is disposed in the tubular portion of the aortic stent;
the aortic valve unit is connected to the proximal portion of the intraventricular stent, said aortic valve unit being disposed at the junction between the intraventricular stent and the aortic stent;
the aortic stent comprises at least two internal branches, preferably three internal branches intended to connect the device to the coronary arteries.
the aortic stent comprises at least two windows, preferably three windows, intended to connect the device to the coronary arteries.
the aortic stent comprises means for connection to other prostheses intended to be inserted into the aorta of a patient;
the intraventricular stent may comprise a notch formed on the periphery of the proximal portion of the intraventricular stent;
the intraventricular stent and the aortic stent consist of a mesh covered with a biocompatible material, such as pericardium.

The invention proposes, according to a second aspect, an assembly comprising a device according to the first aspect of the invention and an envelope, preferably made of felt, said envelope being intended to be wrapped around the aorta of a patient and the aortic stent when said device is deployed in a patient's aorta.

The advantages of the invention are multiple:
the replacement device being in the retracted configuration consisting of a single piece, it allows a complete replacement of the aortic root that is easier than with prostheses in several pieces since this replacement can be done in a single operation;
the presence of a tapered area at the ascending aorta and of the flared portion at the ventricle ensures good proximal and distal sealing between the replacement device and the walls of the aorta to be treated;
the presence of two or three windows or branches allows to maintain the coronary flow to ensure the aortic valve function;
the presence of three windows or branches allows in particular to avoid problems with the orientation of the prosthesis, in particular when it comes to positioning it in emergency situations;
the aortic valve unit that can be placed in several places (in situ or in the ascending aorta) allows adaptation to different clinical situations.

### PRESENTATION OF THE FIGURES

Other features, objects and advantages of the invention will emerge from the description which follows, which is purely illustrative and not limiting, and which must be read in conjunction with the appended drawings in which:

FIG. 1 illustrates a replacement device according to a first embodiment of the invention;

FIG. 2 illustrates an aortic root replacement device according to a first embodiment;

FIG. 3 illustrates the device of FIG. 2 deployed in an aorta;

FIG. 4 illustrates a view of an intraventricular stent of a device according to the first embodiment or the second embodiment;

FIG. 5 illustrates an aortic root replacement device according to a second embodiment;

FIG. 6 illustrates the device of FIG. 4 deployed in an aorta;

FIG. 7 illustrates a replacement device according to a third embodiment;

FIG. 8 illustrates the device of FIG. 7 deployed in an aorta;

FIG. 9 illustrates a sectional view of a replacement device according to the invention.

In all the figures, similar elements bear identical references.

### DETAILED DESCRIPTION

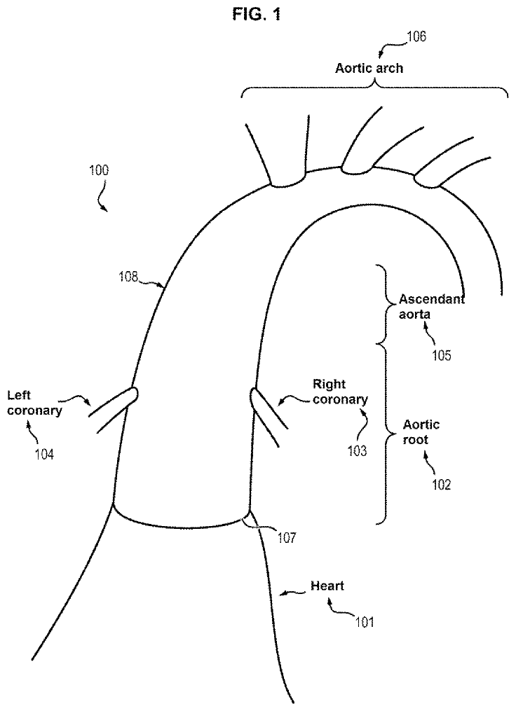

FIG. 1 schematically illustrates the anatomy 100 of the aorta 108 which comprises an aortic root 102 which comprises an aortic valve 107 located at the heart 101 at an aortic annulus which constitutes a junction between the heart 101 and the aorta 102. From the aortic valve 107 emerges the aorta 108 hence the denomination of this area by aortic root 102. In this area, located at the heart 101 emerge the right 104 and left 103 coronaries and the ascending aorta 105 extending along the aortic arch 106.

In what follows, consider an aortic root prosthesis intended to replace the aortic valve and a portion of the ascending aorta (up to an area located upstream of the aortic arch).

In particular, the prosthesis (hereafter aortic root replacement device) which will be described is intended to extend from the heart to an area above the coronary arteries. The notion of “distal” and “proximal” is defined in relation to the aortic valve.

The prosthesis constitutes a self-expanding implantable medical device that can adopt a final position within the aorta which is different from its initial position (before deployment), and which is also different from its position at rest. In particular, the prosthesis is intended to be implanted in a patient by means of a catheter via various vascular accesses (femoral, axillary, humeral, carotid, transapical, etc.).

Thus, the replacement device which will be described is movable between a deployed configuration and a retracted configuration. The retracted position allows the practitioner to implant the device.

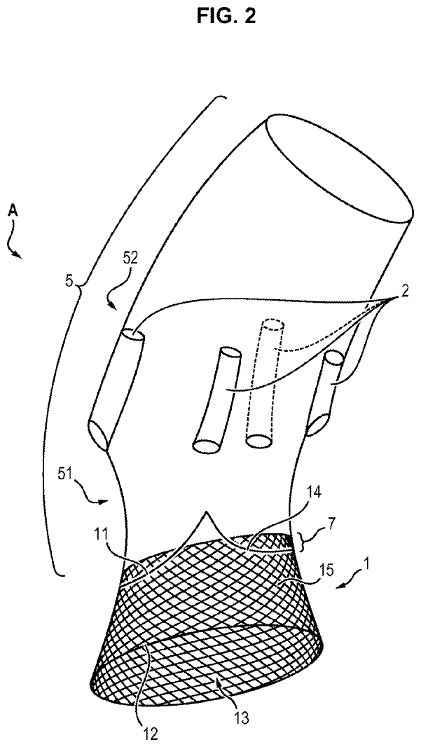

In relation to FIGS. 2 to 8, an aortic root replacement device A, B, C comprises an intraventricular stent 1; an aortic valve unit 7 and an aortic stent 5 extending from the intraventricular stent 1.

Advantageously, the intraventricular stent 1, the aortic valve unit 7 and the aortic stent 5 form a unitary assembly when the device is in the retracted configuration but also in the deployed configuration. In this way, a one-piece aortic root replacement device is obtained when it is implanted, which limits the sealing problems inherent in the junction of several prosthesis portions when it comes to replacing the aortic root. Also, this avoids having to connect the various elements in situ in the patient's body. Furthermore, the aortic root replacement can be performed endovascularly, which avoids major surgery.

The intraventricular stent 1 is intended to be deployed in the ventricle below the aortic valve 107 and advantageously comprises a proximal portion 11 and a distal portion 12 between which the stent 1 is flared such that when it is deployed in the heart 101 (more specifically its ventricle) it is held in position against the internal walls 201, 202 of the ventricle. The distal portion 12 of the stent 1 is therefore located below the aortic valve 107 in the heart and more precisely in the outflow chamber of a cavity of the heart 101.

The distal portion 12 comprises a distal opening 13 and the proximal portion 11 comprises a proximal opening 14 and the stent 1 is flared between the proximal and distal portions.

In other words, it widens from the proximal opening to the distal opening. Indeed, the proximal opening having a diameter smaller than that of the distal opening then, when the stent 1 is inserted into the ventricle when it is deployed (see FIG. 2), the flared portion 15 will deploy to take its place into the ventricle and be held in position therein. The stent 1 therefore has a deployed configuration in the shape of a skirt which prevents the device A from “exiting” the ventricular. Furthermore, the stent 1 in this deployed configuration provides a subvalvular proximal sealing at the aortic annulus and the outflow chamber of the ventricle.

The intraventricular stent 1 consists of a mesh (for example metal mesh) covered with a bio-compatible material such as pericardium. Of course, other materials can be considered. In this way, this material forms a wall which has a sealing to blood and provides a certain rigidity to the stent in addition to that provided by the mesh. Therefore, it forms a conduit for the blood. The aortic stent 5 extends from the aortic valve unit 7 and advantageously consists of a first portion 51 flaring from the intraventricular stent 1 and a tubular portion 52 extending from the first portion 51. The first portion 51 of the stent 5 is tapered. In other words, the section of the aortic stent 5 flares out from the intraventricular stent 1 and has a variable section on this first portion while the tubular portion 52 has a constant section. The aortic stent 5 then has an hourglass shape with the intraventricular stent 1.

Such a shape allows to ensure the sealing of the prosthesis with the wall of the aorta. Indeed, the aortic stent 5 is designed, when it becomes tubular after the flared portion 51, to be in contact with the internal wall of the aorta just above the coronaries.

As for the intraventricular stent 1, the aortic stent 5 consists of a mesh covered with a bio-compatible material such as pericardium. Of course, other materials can be considered. In this way, this material forms a wall which has a sealing to blood and provides a certain rigidity to the stent in addition to that provided by the mesh. Therefore, it forms a conduit for the blood.

The aortic valve unit 7 is connected to the intraventricular stent 1 by means of suture or glue. This aortic valve unit 7 allows to replace the aortic valve while maintaining a natural tricuspid conformation. In particular, the aortic valve unit 7 can either extend from the proximal opening or be inserted inside the intraventricular stent 7.

The aortic valve unit 7 therefore comprises three flaps which open or lower to allow blood to pass into the ventricle (not shown).

Furthermore, the aortic valve unit 7 is made of a bio-compatible material such as pericardium.

The device A, B, C can be connected to other prostheses, endoprosthesis 8 for example. The connection between the prostheses is made by adding another module with a larger diameter and using a certain overlap. It is this second module that will allow connection to the supra-aortic trunks.

The device A, B, C, given both its hourglass shape at the coronaries and its flared shape at the ventricle, allows to obtain sealing at the ventricle but also at the area above the coronaries, with the purpose of excluding the aortic root and the native ascending aorta of the patient from the blood circulation (in particular to reduce the pressure of the blood exerted on the native walls).

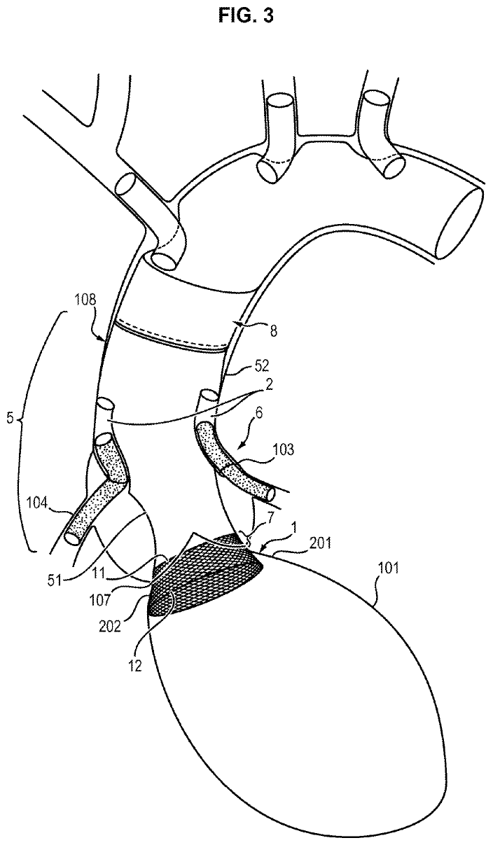

FIG. 2 illustrates an aortic root replacement device A according to a first embodiment and FIG. 3 illustrates the replacement device according to this first embodiment in position in an aorta 108.

According to this embodiment, the aortic valve unit 7 is disposed at the junction between the intraventricular stent 1 and the aortic stent 5. Furthermore, the aortic valve unit 7 is connected to the proximal portion 11 of the intraventricular stent 1 which extends from this proximal portion 11.

Also, the aortic stent 5 comprises in its tubular portion 52 at least two means for connecting the device to the coronary arteries here constituted by internal branches 2.

For example, at least two internal branches 2 allow the coronaries 104, 103 to be perfused. Advantageously, three internal branches 2 are provided. In this case, this allows to ensure, when positioning the device A, that the coronaries are well perfused. Indeed, as can be understood, the device must be correctly oriented with respect to the coronaries. Thus, providing three branches allows to overcome the difficulties of orientation of the device A, each valve sinus being perfused in particular when the latter must be deployed in an emergency situation. Such internal branches 2 facilitate coronary perfusion.

The branches or windows can be pre-loaded by a guide passing therethrough from inside to outside allowing easier access.

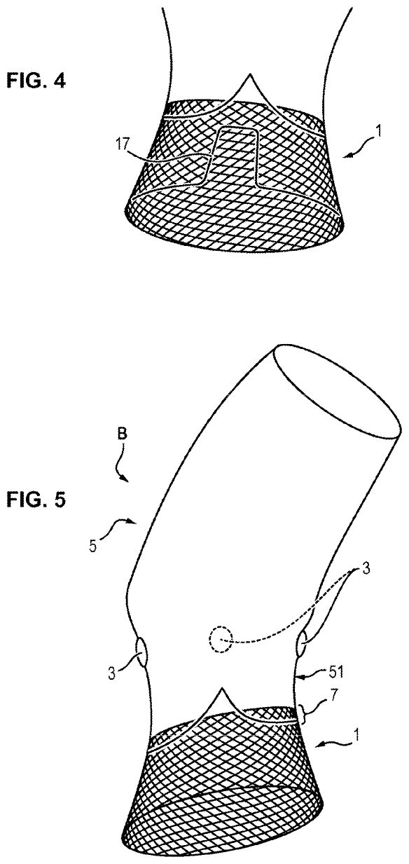

As illustrated in FIG. 4, in addition to each of the embodiments (A, B, C), the intraventricular stent 1 may comprise a notch 17 in order to avoid intracardiac contacts and compressions with the conduction elements of the heart with the purpose of limiting the appearance of post-procedure conduction disorders. The notch 17 is disposed on the periphery of the proximal portion 14 of the intraventricular stent.

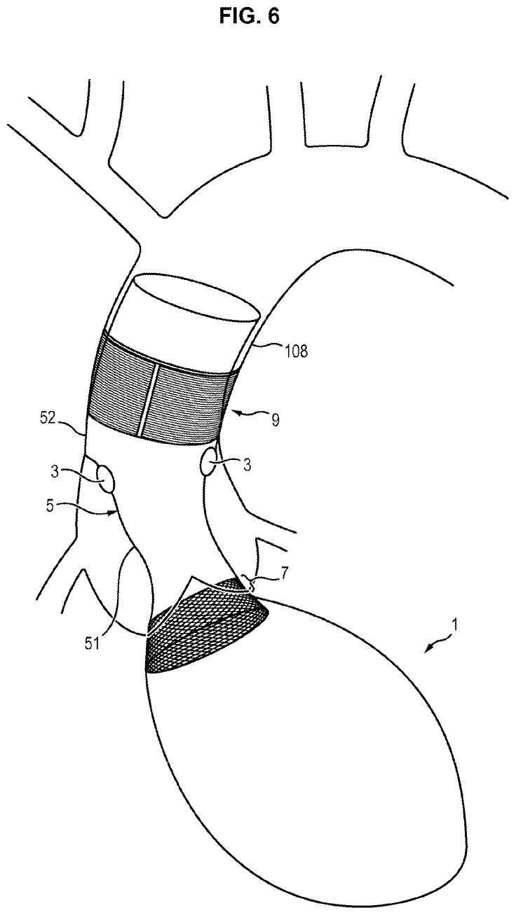

FIG. 5 illustrates an aortic root replacement device B according to a second embodiment and FIG. 6 illustrates the replacement device according to this second embodiment in position in an aorta 108.

This device B according to the second embodiment differs from the device A according to the first embodiment in that the aortic stent 5 comprises two or three means for connecting the device to the coronary arteries here constituted by windows 3 which are practical openings in the aortic stent 5. These windows are preferably disposed in the tapered portion of the stent 5. The windows 3, like the internal branches 2, allow to perfuse the coronaries and facilitate the positioning of the prosthesis in the aorta.

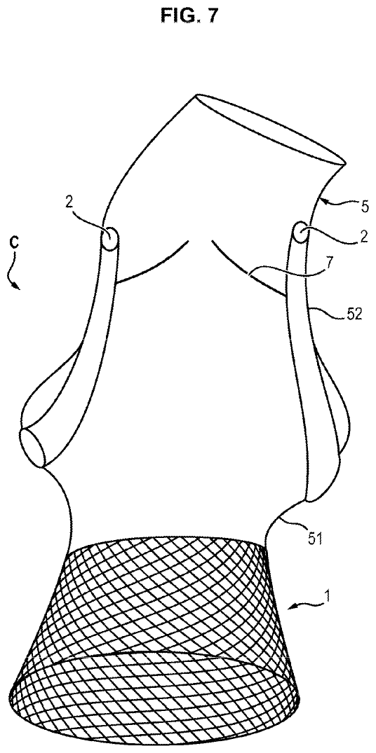

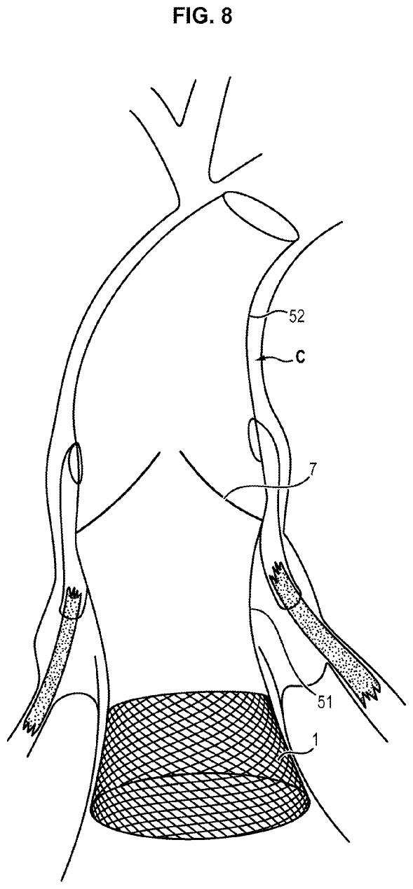

FIG. 7 illustrates an aortic root replacement device C according to a third embodiment and FIG. 8 illustrates the replacement device according to this third embodiment in position in an aorta 108.

This device C according to the third embodiment differs from the devices according to the first and second embodiments (devices A and B) in that the aortic valve unit 7 is disposed in a high position (after the aortic root) in the tubular portion 52 of the intraventricular stent 5 “new ascending aorta”.

This arrangement of the aortic valve applies to cases with windows or branches. The branches (or windows) to the coronaries of this device can be positioned externally or through the aortic valve unit 7.

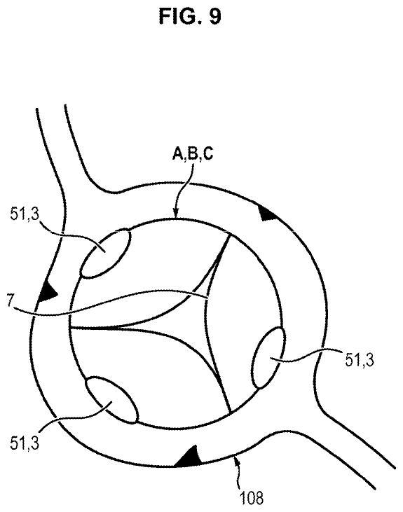

FIG. 9 illustrates a sectional view of the device A, B, C in an aorta 108. In this figure, the tricuspid shape of the aortic valve unit 7 can in particular be distinguished.

The device according to the first, the second embodiment or the third embodiment can advantageously be used with an envelope of felt, or resistant synthetic materials 9 wrapped around the aorta when the device A, B, C is deployed in the aorta. Such a configuration is illustrated in FIG. 6 with the device according to the second embodiment. The use of a felt envelope increases the sealing in the distal portion of the device.

The device can advantageously be implemented with a bag external to the tapered portion 51 which can be filled on demand with a polymeric substance allowing to increase the distal sealing area.

As indicated above, the aortic stent 5 extends from the intraventricular stent 1 and comprises a first portion 51 extending from the intraventricular stent 1 and a tubular portion 52 extending from the first portion 51.

In order to suit the different mechanical stresses of the areas in which the device is disposed, the latter consists of several meshes which have different mechanical properties. In particular, it is the radial force of the mesh that differs. Radial force means a resistance of the mesh in a radial direction of the device with respect to its longitudinal axis. Indeed, the different portions are substantially cylindrical at least per piece so that they all comprise an axis of revolution from which the radial direction is defined.

Advantageously, the first portion 51 of the stent 5 consists of a first mesh, the tubular portion 52 consists of a second mesh, the intraventricular stent 1 consists of a third mesh, the first mesh, the second mesh and the third mesh having different mechanical properties.

The first mesh advantageously has a radial force greater than the radial force of the second mesh, the first mesh being configured to provide a flexibility necessary for the curvature of the aortic root in which the first portion 51 is intended to be positioned. Preferably, the first mesh comprises a Z mesh.

The second mesh is advantageously configured to allow to obtain different lengths of the tubular portion 52. It is preferably a spiral mesh. Such a conformation is adaptable and allows, with an adapted launcher, to compress or stretch the stent 5, thus allowing to obtain different lengths for the tubular portion 52 in order to best adapt to the anatomy of the patient.

The third mesh is advantageously a tighter mesh than the first and second meshes in order to ensure sealing in the area where it is disposed. It is a quadrangular tight mesh. Indeed, in this area, it is essential to have a strong sealing.

1. An aortic root replacement device movable between a deployed configuration and a retracted configuration, the aortic root replacement device comprising
an intraventricular stent comprising a proximal portion and a distal portion, the intraventricular stent being flared from the proximal portion towards the distal portion such that when the aortic root replacement device is deployed in the ventricle below the aortic valve aortic root replacement device is held in position in the ventricle;
an aortic valve unit;
an aortic stent extending from the intraventricular stent;
the intraventricular stent, the aortic valve unit and the aortic stent forming a unitary assembly in retracted configuration and in deployed configuration, the aortic stent comprising at least three means for connecting the aortic root replacement device to the coronary arteries.

2. The aortic root replacement device according to claim 1, wherein the aortic stent extends from the intraventricular stent and comprises a first portion extending from the intraventricular stent and a tubular portion extending from the first portion.

3. The aortic root replacement device according to claim 2, wherein the aortic valve unit is disposed in the tubular portion of the aortic stent.

4. The aortic root replacement device according to claim 2, wherein the first portion consists of a first mesh, the tubular portion consists of a second mesh, the intraventricular stent consists of a third mesh, the first mesh, the second mesh and the third mesh having different mechanical properties.

5. The aortic root replacement device according to claim 4, wherein the first mesh has a radial force greater than the radial force of the second mesh, the first mesh being configured to provide a flexibility necessary for the curvature of the aortic root in which the first portion is intended to be positioned, the second mesh being configured to allow to obtain different lengths of said tubular portion.

6. The aortic root replacement device according to claim 4, wherein the third mesh is a tighter mesh than the first and second meshes in order to ensure sealing in the area where it is disposed.

7. The aortic root replacement device according to claim 1, wherein the aortic valve unit is connected to the proximal portion of the intraventricular stent, the aortic valve unit being disposed at the junction between the intraventricular stent and the aortic stent.

8. The aortic root replacement device according to claim 1, wherein the means for connecting the aortic root replacement device to the coronary arteries comprise three internal branches intended to connect the aortic root replacement device to the coronary arteries.

9. The aortic root replacement device according to claim 1, wherein the means for connecting the aortic root replacement device to the coronary arteries comprise three windows, intended to connect the aortic root replacement device to the coronary arteries.

10. The aortic root replacement device according to claim 1, wherein the aortic stent comprises means for connecting the aortic root replacement device to other prostheses intended to be inserted into the aorta of a patient.

11. The aortic root replacement device according to claim 1, wherein the intraventricular stent comprises a notch formed on the periphery of the proximal portion of the intraventricular stent.

12. The aortic root replacement device according to claim 1, wherein the intraventricular stent and the aortic stent consist of a mesh covered with a biocompatible material, such as pericardium.

13. An assembly comprising a device according to claim 1 and an envelope, the envelope being intended to be wrapped around the aorta of a patient and the aortic stent when the aortic root replacement device is deployed in the aorta of a patient.

14. The assembly according to claim 13, wherein the envelope is made of felt.
